(19) United States
(12) Patent Application Publication
(10) Pub. No.: US 2025/0369942 A1
(43) Pub. Date: Dec. 4, 2025

(54) CONTROLLED ENVIRONMENT SYSTEM FOR STANDARDIZING INITIAL CONDITIONS IN CO2 SORBENT CHARACTERIZATION

(71) Applicants: Kacie NIIMOTO, Mesa, AZ (US); Klaus Stephan LACKNER, Longmont, CO (US); Matthew GREEN, Phoenix, AZ (US)

(72) Inventors: Kacie NIIMOTO, Mesa, AZ (US); Klaus Stephan LACKNER, Longmont, CO (US); Matthew GREEN, Phoenix, AZ (US)

(73) Assignee: Arizona Board of Regents on Behalf of Arizona State University, Scottsdale, AZ (US)

(21) Appl. No.: 19/221,375

(22) Filed: May 28, 2025

Related U.S. Application Data
(60) Provisional application No. 63/652,634, filed on May 28, 2024.

(51) Int. Cl. G01N 33/00

(52) U.S. Cl. CPC G01N 33/0073; G01N 33/0016; G01N 33/0019; G01N 33/004

(57) ABSTRACT

A controlled environment system for standardizing initial conditions in CO₂ sorbent characterization is disclosed. The system includes an enclosure that is sealable and a humidity control subsystem having a vessel inside the enclosure, with the vessel containing a saturated salt solution. The system also includes a gas control subsystem having a CO₂ sensor within the enclosure and an electric valve through which the interior of the enclosure is in fluidic communication with a gas supply. The system includes a microcontroller communicatively coupled to the CO₂ sensor and the electric valve, and configured to function in a gas control-feedback loop, driving the electric valve in response to the CO₂ sensor. The saturated salt solution is chosen and the gas control-feedback loop of the gas control subsystem is configured such that a characterization loading condition is established and maintained within the interior of the enclosure while the enclosure is sealed.

### RELATED APPLICATIONS

This application claims the benefit of U.S. provisional patent application 63/652,634, filed May 28, 2024 titled “Controlled Environment System for Standardizing Initial Conditions in CO₂ Sorbent Characterization,” the entirety of the disclosure of which is hereby incorporated by this reference.

### STATEMENT REGARDING FEDERALLY SPONSORED RESEARCH

This invention was made with government support under DE-SC0023343 awarded by the Department of Energy. The government has certain rights in the invention.

### TECHNICAL FIELD

Aspects of this document relate generally to controlled environments for material characterization.

### BACKGROUND

The need for technologies to remove carbon dioxide from ambient air has been well established. The average CO₂ concentration in the atmosphere was 415 ppm in June 2021, which has been linked to elevated average global temperatures, extreme weather, wildfires, and more. The Intergovernmental Panel on Climate Change's 2021 report predicts a 1.5° C. rise in global temperature within the next two decades, assuming global policymakers aggressively reduce emissions. Without intervention, an average temperature rise of 4.4° C. is possible, leading to catastrophic results. Current CO₂ emissions are projected to reach 40 GT CO₂/year.

In addition to conservation, reduced-carbon processes, and on-site capture efforts, a significant amount of carbon dioxide will need to be removed from the atmosphere to avoid a looming climate change crisis. Negative emission technologies are a critical technological solution needed to reduce emissions and lead to a net-zero emission scenario. Capture of carbon dioxide from ambient air at an affordable price could become a critical tool in managing the anthropogenic carbon cycle.

A promising technology that is well adapted for capturing dilute atmospheric carbon dioxide in an energy efficient manner is Direct Air Capture (DAC). Out of the many available DAC technologies, sorption-based methods show promising results. Chemisorption and physisorption are the two broadly classified mechanisms for CO₂ sorption. The regeneration/desorption can be achieved by altering different operating conditions including, but not limited to, temperature (temperature-swing) and humidity (moisture-swing).

Widespread adoption of DAC technology will depend on their ability to operate within a tight energy budget. It is very important that the best sorbent material is chosen for a particular use environment. However, when characterizing the performance of sorbent materials, it can be very challenging to obtain reproducible results due to the highly variable humidities and temperatures in the lab. Without a standardized testing procedure with repeatable starting conditions, meaningful comparison and selection of the sorbent materials will be difficult.

### SUMMARY

According to one aspect, a controlled characterization environment system includes an enclosure that is sealable and includes an interior, and a humidity control subsystem having a vessel inside the enclosure, with the vessel containing a saturated salt solution. The system also includes a gas control subsystem having a CO₂ sensor within the enclosure and an electric valve through which the interior of the enclosure is in fluidic communication with a gas supply. The system also includes a microcontroller communicatively coupled to the CO₂ sensor and the electric valve, with the microcontroller configured to function in a gas control-feedback loop, driving the electric valve in response to the CO₂ sensor. The saturated salt solution of the humidity control subsystem is chosen and the gas control-feedback loop of the gas control subsystem is configured such that a characterization loading condition is established and maintained within the interior of the enclosure while the enclosure is sealed.

Particular embodiments may comprise one or more of the following features. The saturated salt solution may include at least one of ammonium nitrate, ammonium sulphate, lithium chloride, magnesium chloride, magnesium nitrate, natrium chloride, potassium sulphate, potassium nitrate, potassium chloride, potassium acetate, potassium hydroxide, sodium chloride, sodium nitrite, and sodium dichromate. The humidity control subsystem may be electricity-independent. The gas supply may be an air line, with the air line putting the interior of the enclosure in fluidic communication with an ambient atmosphere through the electric valve. The gas supply may be one of an air compressor and a gas cylinder. The system may further include a temperature control subsystem that may include a thermal conditioner and a thermal sensor. The thermal conditioner and thermal sensor may be communicatively coupled to the microcontroller. The microcontroller may be configured to provide a thermal control-feedback loop, driving the thermal conditioner in response to the thermal sensor. The characterization loading condition may include a CO₂ concentration that is between 400-500 ppm.

According to another aspect of the disclosure, a controlled characterization environment system includes an enclosure that is sealable and includes an interior. The system also includes a humidity control subsystem having a vessel inside the enclosure, with the vessel containing a saturated salt solution. The system further includes a gas control subsystem having a CO₂ sensor within the enclosure and an electric valve through which the interior of the enclosure is in fluidic communication with a gas supply, and a temperature control subsystem having a thermal conditioner in thermal contact with the interior of the enclosure and a thermal sensor within the enclosure. The system includes a microcontroller communicatively coupled to the CO₂ sensor, the electric valve, the thermal conditioner, and the thermal sensor. The microcontroller is configured to provide a gas control-feedback loop, driving the electric valve in response to the CO₂ sensor. The microcontroller is further configured to provide a thermal control-feedback loop, driving the thermal conditioner in response to the thermal sensor. The saturated salt solution of the humidity control subsystem is chosen and the gas control-feedback loop and thermal control-feedback loop are configured such that a characterization loading condition is established and maintained within the interior of the enclosure while the enclosure is sealed.

Particular embodiments may comprise one or more of the following features. The saturated salt solution may include at least one of ammonium nitrate, ammonium sulphate, lithium chloride, magnesium chloride, magnesium nitrate, natrium chloride, potassium sulphate, potassium nitrate, potassium chloride, potassium acetate, potassium hydroxide, sodium chloride, sodium nitrite, and sodium dichromate. The gas supply may be an air line, with the air line putting the interior of the enclosure in fluidic communication with an ambient atmosphere through the electric valve. The gas supply may be one of an air compressor and a gas cylinder. The characterization loading condition may include a CO₂ concentration that is between 400-500 ppm.

According to yet another aspect of the disclosure, a controlled characterization environment system includes an enclosure that is sealable and having an interior, a humidity control subsystem, and a gas control subsystem having a CO₂ sensor within the enclosure and an electric valve through which the interior of the enclosure is in fluidic communication with a gas supply. The system also includes a microcontroller communicatively coupled to the CO₂ sensor and the electric valve, with the microcontroller configured to function in a gas control-feedback loop, driving the electric valve in response to the CO₂ sensor. The humidity control subsystem and the gas control-feedback loop are configured such that a characterization loading condition is established and maintained within the interior of the enclosure while the enclosure is sealed.

Particular embodiments may comprise one or more of the following features. The humidity control subsystem may include a vessel inside the enclosure, with the vessel containing a saturated salt solution. The saturated salt solution may include at least one of ammonium nitrate, ammonium sulphate, lithium chloride, magnesium chloride, magnesium nitrate, natrium chloride, potassium sulphate, potassium nitrate, potassium chloride, potassium acetate, potassium hydroxide, sodium chloride, sodium nitrite, and sodium dichromate. The humidity control subsystem may include a bubbler. The gas supply may be an air line, with the air line putting the interior of the enclosure in fluidic communication with an ambient atmosphere through the electric valve. The gas supply may be one of an air compressor and a gas cylinder. The system may further include a temperature control subsystem that may include a thermal conditioner and a thermal sensor. The thermal conditioner and thermal sensor may be communicatively coupled to the microcontroller. The microcontroller may be configured to provide a thermal control-feedback loop, driving the thermal conditioner in response to the thermal sensor. The characterization loading condition may include a CO₂ concentration that is between 400-500 ppm.

Aspects and applications of the disclosure presented here are described below in the drawings and detailed description. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112(f). Thus, the use of the words “function,” “means” or “step” in the Detailed Description or Description of the Drawings or claims is not intended to somehow indicate a desire to invoke the special provisions of 35 U.S.C. § 112(f), to define the invention. To the contrary, if the provisions of 35 U.S.C. § 112(f) are sought to be invoked to define the inventions, the claims will specifically and expressly state the exact phrases “means for” or “step for”, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112(f). Moreover, even if the provisions of 35 U.S.C. § 112(f) are invoked to define the claimed aspects, it is intended that these aspects not be limited only to the specific structure, material or acts that are described in the preferred embodiments, but in addition, include any and all structures, materials or acts that perform the claimed function as described in alternative embodiments or forms of the disclosure, or that are well known present or later-developed, equivalent structures, material or acts for performing the claimed function.

The foregoing and other aspects, features, and advantages will be apparent to those artisans of ordinary skill in the art from the DESCRIPTION and DRAWINGS, and from the CLAIMS.

### BRIEF DESCRIPTION OF THE DRAWINGS

The disclosure will hereinafter be described in conjunction with the appended drawings, where like designations denote like elements, and:

FIG. 1A is a schematic view of a controlled characterization environment system;

FIG. 1B is a schematic view of another embodiment of a controlled characterization environment system; and

FIG. 2 is a perspective view of an enclosure for a controlled characterization environment system.

### DETAILED DESCRIPTION

This disclosure, its aspects and implementations, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

While this disclosure includes a number of embodiments in many different forms, there is shown in the drawings and will herein be described in detail particular embodiments with the understanding that the present disclosure is to be considered as an exemplification of the principles of the disclosed methods and systems, and is not intended to limit the broad aspect of the disclosed concepts to the embodiments illustrated.

The need for technologies to remove carbon dioxide from ambient air has been well established. The average CO₂ concentration in the atmosphere was 415 ppm in June 2021, which has been linked to elevated average global temperatures, extreme weather, wildfires, and more. The Intergovernmental Panel on Climate Change's 2021 report predicts a 1.5° C. rise in global temperature within the next two decades, assuming global policymakers aggressively reduce emissions. Without intervention, an average temperature rise of 4.4° C. is possible, leading to catastrophic results. Current CO₂ emissions are projected to reach 40 GT CO₂/year.

In addition to conservation, reduced-carbon processes, and on-site capture efforts, a significant amount of carbon dioxide will need to be removed from the atmosphere to avoid a looming climate change crisis. Negative emission technologies are a critical technological solution needed to reduce emissions and lead to a net-zero emission scenario. Capture of carbon dioxide from ambient air at an affordable price could become a critical tool in managing the anthropogenic carbon cycle.

A promising technology that is well adapted for capturing dilute atmospheric carbon dioxide in an energy efficient manner is Direct Air Capture (DAC). Out of the many available DAC technologies, sorption-based methods show promising results. Chemisorption and physisorption are the two broadly classified mechanisms for CO₂ sorption. The regeneration/desorption can be achieved by altering different operating conditions including, but not limited to, temperature (temperature-swing) and humidity (moisture-swing).

Widespread adoption of DAC technology will depend on their ability to operate within a tight energy budget. It is very important that the best sorbent material is chosen for a particular use environment. However, when characterizing the performance of sorbent materials, it can be very challenging to obtain reproducible results due to the highly variable humidities and temperatures in the lab. Without a standardized testing procedure with repeatable starting conditions, meaningful comparison and selection of the sorbent materials will be difficult.

Contemplated herein is a controlled environment system to standardize conditions for characterizing sorbent or other materials. The controlled characterization environment system (hereinafter “CCE system” or “system”) utilizes an automated control system to stabilize sorbent materials to standard loading states of carbon dioxide and water vapor. Allowing sorbent materials to reach an equilibrium within the same, specific environment ensures all the sorbent materials start at the same initial conditions. This makes it possible to make comparisons and draw accurate conclusions on their direct air capture performance.

According to various embodiments, the system contemplated herein is able to control CO₂, humidity, and temperature at a low cost. According to various embodiments, the system uses saturated salt solutions and/or a bubbler to control humidity and an automated gas valve to control CO₂ concentration in the enclosure. The valve is connected to a gas source providing CO₂ (e.g., atmospheric CO₂ at ˜400 ppm air, etc.). In some embodiments, temperature may also be controlled using simple heating or cooling elements. This simplified approach makes the system low-cost, easy to produce and use, as well as effective and reproducible. Since many carbon capture sorbents are highly sensitive to CO₂, humidity, and temperature, this system will aid in the development of carbon capture materials by standardizing performance and material characterization measurements.

It should be noted that while much of the discussion of the contemplated system is done in the context of characterizing sorbent materials for use in direct air capture, the system may be used for other purposes. According to various embodiments, the contemplated system may be adapted for use in any application that requires consistent levels of CO₂, humidity, and temperature levels. Examples include, but are not limited to, standardized characterization of materials and standardized characterization of sensors or other observation devices. Other embodiments of the system may be adapted to maintain consistent levels of other atmospheric gases, or the concentration of a gas provided from a supply.

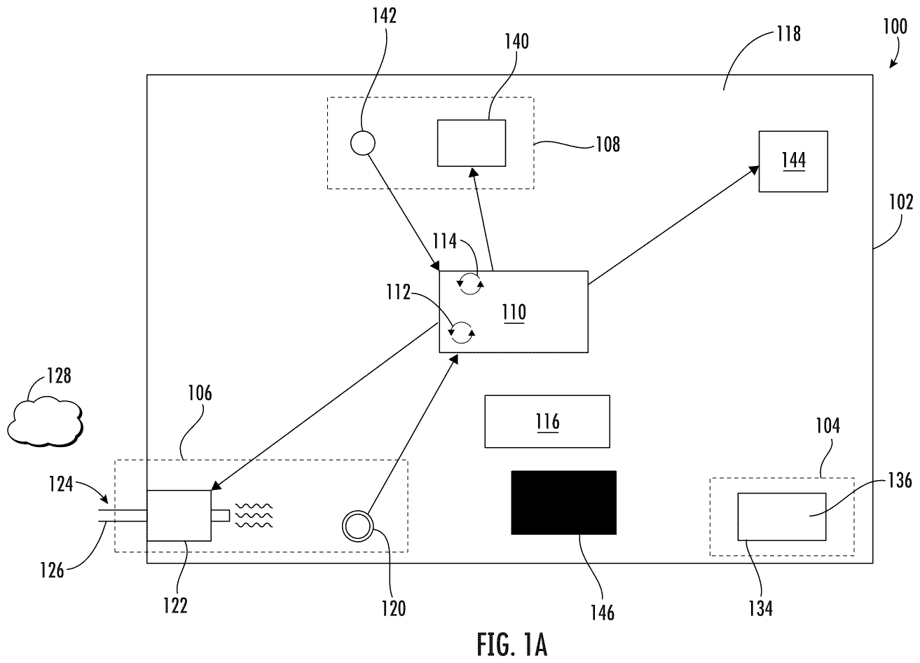

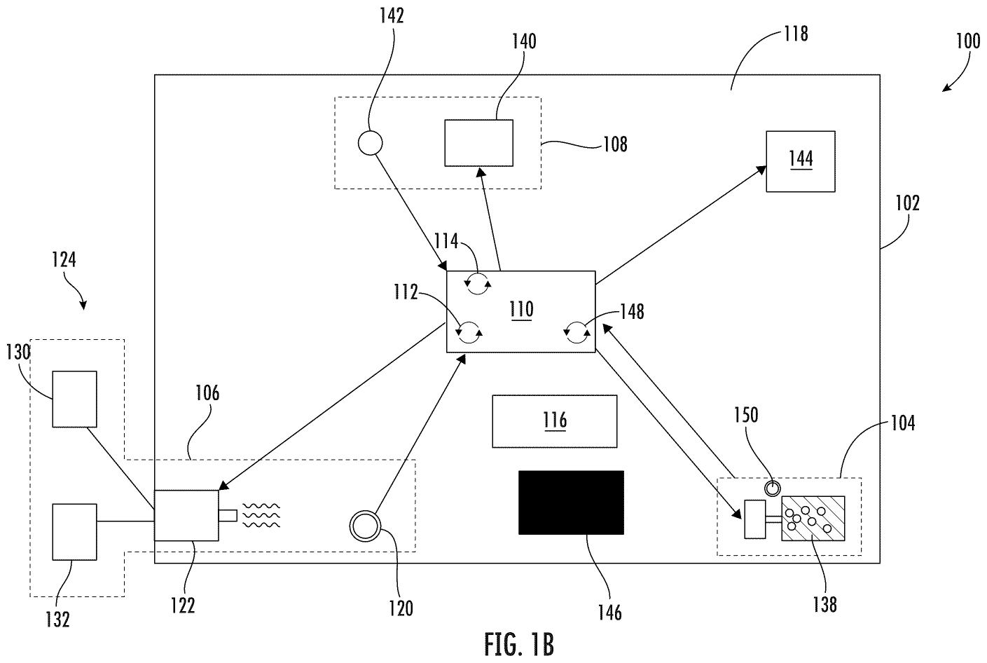

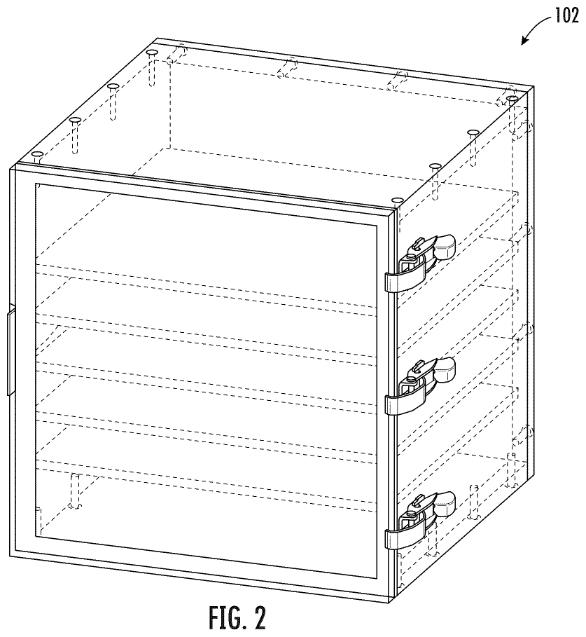

FIGS. 1A and 1B are schematic views of different non-limiting examples of a controlled characterization environment system 100. According to various embodiments, the system is made up of an enclosure 102 that houses a humidity control subsystem 104 and a gas control subsystem 106. In some embodiments, the enclosure 102 may also comprise a temperature control subsystem 108. Advantageously, each of these subsystems is effective, inexpensive, and easy to operate. Each will be discussed in turn. The enclosure 102 will be discussed further in the context of FIG. 2.

The controlled characterization environment system 100 comprises a humidity control subsystem 104 that maintains the relative humidity within interior 118 of the enclosure 102 within a desired range. Advantageously, the contemplated system 100 can maintain environments with humidity ranging from low (e.g., 3% humidity, etc.) to high (e.g., over 90% humidity, etc.), according to various embodiments. The controlled characterization system 100 is able to maintain a particular humidity level with a high degree of accuracy (e.g., within 1%), once conditions have stabilized within the chamber.

In some embodiments, including the non-limiting example shown in FIG. 1A, the humidity control subsystem 104 maintains relative humidity levels using one or more vessels 134 containing saturated salt solutions 136. Humidity is controlled by exposing saturated salt solutions 136 within the enclosure 102. Advantageously, this accomplishes consistent control of the humidity, yet does not require a complex control system. In fact, in some embodiments, humidity control subsystem 104 may be entirely electricity-independent, operating without any control system while still maintaining a desired relative humidity set point within the enclosure 102.

According to various embodiments, the saturated salt solutions 136 are loaded into one or more open vessels 134 (e.g., glass petri dishes, plastic trays, etc.) within the enclosure 102. In some embodiments the salt solution vessels 134 may be located at the bottom of the enclosure 102. In other embodiments, the vessel 134 or vessels 134 may be located anywhere else within the enclosure 102 (e.g., on shelves or surfaces holding samples 146 being stabilized). As an option, in some embodiments, one or more circulation fans 144 may be utilized to mix the air within the enclosure 102 such that the humidity equilibrium is reached more quickly.

Particular levels of humidity are dependent upon the type of salt used in the humidity control subsystem 104, according to various embodiments. For example, if the vessels 134 contained a saturated salt solution 136 of potassium nitrate at 25° C., the corresponding humidity level within the enclosure 102 would be about 97% relative humidity. If vessels 134 contained a saturated potassium acetate solution 136 under the same conditions, the corresponding humidity level within the enclosure 102 would be about 23% relative humidity. Therefore, for a desired humidity set point, an appropriate salt species must be chosen. Examples of saturated salt solutions 136 that may be used in the CCE system 100 include, but are not limited to, ammonium nitrate, ammonium sulphate, lithium chloride, magnesium chloride, magnesium nitrate, natrium chloride, potassium sulphate, potassium nitrate, potassium chloride, potassium acetate, potassium hydroxide, sodium chloride, sodium nitrite, and sodium dichromate.

In other embodiments, the humidity control subsystem 104 may use other humidity manipulation methods and devices. For example, in some embodiments, including the non-limiting example shown in FIG. 1B, the humidity control subsystem 104 may comprise a bubbler 138, which introduces humidity by bubbling gas up through water or some other solution. Unlike the electricity-independent embodiment shown in FIG. 1A, the bubbler 138 of FIG. 1B requires a control system. The use of control-feedback loops and other control methods will be discussed below, in the context of this and other subsystems of the CCE system 100.

The controlled characterization environment system 100 comprises a gas control subsystem 106 that maintains the concentration of a gas (e.g., CO₂, etc.) in the interior 118 of the enclosure 102 within a desired range, such as ambient levels (i.e., 400-500 ppm CO₂). Carbon capture sorbent materials can either sorb or desorb CO₂ under particular temperature, humidity, and CO₂ conditions. Therefore, when sorbent samples 146 are sealed inside the enclosure 102 at specified conditions, a loss or increase of CO₂ can occur as they stabilize. According to various embodiments, the gas control subsystem 106 is configured to maintain the CO₂ levels of the enclosure 102 at ambient levels despite the introduction or removal of CO₂ due to sorbent materials stabilizing within the sealed enclosure 102.

According to various embodiments, the gas control subsystem 106 comprises a CO₂ sensor 120 within the enclosure 102 and an automated or electric valve 122 connected to a gas supply 124. Put differently, the interior 118 of the enclosure 102 is in fluidic contact with the gas supply 124 through the electric valve 122 (when the electric valve 122 is not closed), according to various embodiments. In the context of the present description and the claims that follow, an electric valve 122 is a valve that can be opened and closed using an electrical signal. In some embodiments, the electric valve 122 may operate based on a control signal while receiving the power to move from another source. In other embodiments, the electric valve 122 may open or close when powered, depending on where the power is received. In some embodiments, the electric valve 122 may switch between an open state and a closed state, while in other embodiments the electric valve 122 may be configured to partially open to different degrees.

In some embodiments, the CO₂ sensor 120 and electric valve 122 operate together as part of a gas control-feedback loop 112. In some embodiments, including the non-limiting examples shown in FIGS. 1A and 1B, the gas control-feedback loop 112 may be implemented using a microcontroller 110. The CO₂ sensor 120 is able to measure the CO₂ concentration in units of parts per million (by volume). Examples of CO₂ sensors 120 include, but are not limited to, non-dispersive infrared (NDIR) sensors, electrochemical sensors, thermal conductivity, chromatographic sensors, and the like.

Various gas supplies 124 may be used in conjunction with the gas control subsystem 106. In some embodiments, the gas supply 124 may be the ambient atmosphere 128. As shown in the non-limiting example of FIG. 1A, the interior of the enclosure 102 (when sealed) may be in fluidic communication with the ambient atmosphere 128 outside the enclosure 102 through an air line 126 that is controlled by the electric valve 122.

In other embodiments, including the non-limiting example shown in FIG. 1B, the gas supply 124 may operate above ambient air pressure. Examples of pressurized gas supplies 124 include, but are not limited to, an air compressor 130 pressurizing ambient atmosphere 128 and/or a gas cylinder 132 containing 400 ppm CO₂ in air. As an option, a pressure regulator may also be used. In still other embodiments the system may be adapted for use in creating a controlled environment for the purposes of characterizing something other than atmospheric carbon dioxide DAC materials. In some embodiments, the gas control subsystem 106 may comprise a gas supply 124 providing a gas, atmospheric or otherwise, other than carbon dioxide.

In some embodiments, the contemplated CCE system 100 is operated at the ambient temperature (e.g., whatever temperature the laboratory is maintained at). In other embodiments, the system 100 may comprise a temperature control subsystem 108 to maintain a desired temperature within the enclosure 102 as the samples 146 stabilize. This may be of use in cases where the intended use environment (and thus, intended characterization environment) is a different temperature than the ambient laboratory temperature or in cases where the temperature in the surroundings varies more than the acceptable range.

As shown, the temperature control subsystem 108 may comprise a thermal conditioner 140 and a thermal sensor 142. In the context of the present description and the claims that follow, a thermal conditioner 140 is an electrical device that is able to increase and/or decrease the temperature of its surroundings. Examples include, but are not limited to, a refrigeration unit, a Peltier cooler, a heat pump, and the like. The thermal conditioner 140 is controllable electrically, and both it and at least one thermal sensor 142 are communicatively coupled to a control device such as a microcontroller 110 (e.g., Arduino, Raspberry Pi, ESP32, etc.).

According to various embodiments, a control device such as a microcontroller 110 may be used to drive various subsystems to maintain their particular portion of the environment within the enclosure to be within a desired range. For example, in some embodiments, including the non-limiting examples shown in FIGS. 1A and 1B, a microcontroller 110 may be communicatively coupled to the CO₂ sensor 120 and the electric valve 122 of the gas control subsystem 106, with the microcontroller 110 configured to function in a gas control-feedback loop 112, driving the electric valve 122 to open or close in response to readings from the CO₂ sensor 120. Furthermore, in some embodiments, the thermal conditioner 140 and thermal sensor 142 of a temperature control subsystem 108 may be communicatively coupled to the microcontroller 110, which is configured to function in a thermal control-feedback loop 114, driving the thermal conditioner 140 in response to readings from the thermal sensor 142.

In some embodiments, the humidity control subsystem 104 may be electricity-independent (e.g., using saturated salt solution 136 in open vessels 134, etc.) and not require any control systems. In other embodiments, including the non-limiting example shown in FIG. 1B, the humidity control subsystem 104 may be electric, and able to be controlled by a microcontroller 110. As shown in FIG. 1B, in a specific embodiment, the humidity control subsystem 104 may comprise a bubbler 138 and a humidity sensor 150. The bubbler 138 and the humidity sensor 150 are communicatively coupled to the microcontroller 110, which is configured to function in a humidity control-feedback loop 148, driving the bubbler 138 in response to readings from the humidity sensor 150.

In embodiments comprising more than one of the discussed control-feedback loops, all of the loops may be implemented by the same control device (e.g., microcontroller 110). In other embodiments, the CCE system 100 may be more modular, with each subsystem employing a control-feedback loop also comprising its own control device.

According to various embodiments, after a sample 146 or samples 146 (e.g., sorbent materials, etc.) have been placed inside the contemplated controlled characterization environment system 100 and the enclosure has been sealed, the various subsystems adjust to achieve and maintain a specific environment (i.e., humidity, temperature, gas concentration) that they were configured to target. This set of targeted characteristics is the characterization loading condition 116, and describes what the sample 146 will have stabilized to after spending sufficient time within the CCE system 100. According to various embodiments, the humidity control subsystem 104, the gas control subsystem 106, and the temperature control subsystem 108 (if present) are configured (e.g., choice of saturated salt solution 136, parameters for a gas control-feedback loop 112, etc.) such that the characterization loading condition 116 is established and maintained within the interior 118 of the enclosure 102 while the enclosure 102 is sealed.

FIG. 2 is a perspective view of a non-limiting example of an enclosure 102 for a controlled characterization environment system 100. In some embodiments, the enclosure 102 may be composed of acrylic. In other embodiments, the enclosure 102 may be composed of any other material known in the art. As shown, the enclosure 102 can open to receive samples 146 (e.g., sorbent materials) and other items such as vessels 134 containing saturated salt solutions 136, and is then able to be sealed. As shown, in some embodiments, the enclosure 102 interior may comprise one or more shelves to hold the samples 146 that are being stabilized prior to characterization.

According to various embodiments, the enclosure 102 is sealable, meaning able to be sealed sufficient enough that the desired environment (i.e., characterization loading condition 116) can be maintained over the time range of the stabilization by the various subsystems. In some embodiments, the closed enclosure 102 may be airtight. In other embodiments, the enclosure 102 may have a looser seal. For example, an airtight enclosure 102 may not be needed, depending on the construction and the amount of gas exchange.

The enclosure 102 provides a controlled, stable environment for stabilizing samples 146 to a predefined initial state (i.e., characterization loading condition 116), in anticipation of their characterization. Carbon capture performance is often measured in terms of the amount of CO₂ captured per mass of sorbent (i.e., CO₂ uptake capacity) and the rate of CO₂ capture. According to various embodiments, prior to performance testing, the sorbent materials may be vacuum-dried overnight to obtain the true dry weight that will be used in the calculations of CO₂ uptake capacity. From there, the samples 146 are maintained in a controlled environment created by the CCE system 100 where the CO₂, humidity, and temperature are constant (within a tolerance) in order to start testing at the same loading conditions of carbon dioxide and water. As a specific example, in one embodiments, sorbents are stored within the enclosure 102 for at least 48 hours before testing for direct air capture performance.

It will be understood that implementations are not limited to the specific components disclosed herein, as virtually any components consistent with the intended operation of a controlled characterization environment system may be utilized. Accordingly, for example, although particular systems, methods, and/or devices for a controlled environment system for standardizing initial conditions in CO₂ sorbent characterization may be disclosed, such components may comprise any shape, size, style, type, model, version, class, grade, measurement, concentration, material, weight, quantity, and/or the like consistent with the intended operation of a controlled characterization environment system for standardizing initial conditions in CO₂ sorbent characterization may be used. In places where the description above refers to particular implementations of a controlled characterization environment system, it should be readily apparent that a number of modifications may be made without departing from the spirit thereof and that these implementations may be applied to other environmental control systems and methods.

What is claimed is:

1. A controlled characterization environment system, comprising:
an enclosure that is sealable and comprises an interior;
a humidity control subsystem comprising a vessel inside the enclosure, with the vessel containing a saturated salt solution;
a gas control subsystem comprising a CO₂ sensor within the enclosure and an electric valve through which the interior of the enclosure is in fluidic communication with a gas supply; and
a microcontroller communicatively coupled to the CO₂ sensor and the electric valve, with the microcontroller configured to function in a gas control-feedback loop, driving the electric valve in response to the CO₂ sensor;
wherein the saturated salt solution of the humidity control subsystem is chosen and the gas control-feedback loop of the gas control subsystem is configured such that a characterization loading condition is established and maintained within the interior of the enclosure while the enclosure is sealed.

2. The controlled characterization environment system of claim 1, wherein the saturated salt solution comprises at least one of ammonium nitrate, ammonium sulphate, lithium chloride, magnesium chloride, magnesium nitrate, natrium chloride, potassium sulphate, potassium nitrate, potassium chloride, potassium acetate, potassium hydroxide, sodium chloride, sodium nitrite, and sodium dichromate.

3. The controlled characterization environment system of claim 1, wherein the humidity control subsystem is electricity-independent.

4. The controlled characterization environment system of claim 1, wherein the gas supply is an air line, with the air line putting the interior of the enclosure in fluidic communication with an ambient atmosphere through the electric valve.

5. The controlled characterization environment system of claim 1, wherein the gas supply is one of an air compressor and a gas cylinder.

6. The controlled characterization environment system of claim 1, further comprising a temperature control subsystem comprising a thermal conditioner and a thermal sensor, wherein the thermal conditioner and thermal sensor are communicatively coupled to the microcontroller, and wherein the microcontroller is configured to provide a thermal control-feedback loop, driving the thermal conditioner in response to the thermal sensor.

7. The controlled characterization environment system of claim 1, wherein the characterization loading condition comprises a CO₂ concentration that is between 400-500 ppm.

8. A controlled characterization environment system, comprising:
an enclosure that is sealable and comprises an interior;
a humidity control subsystem comprising a vessel inside the enclosure, with the vessel containing a saturated salt solution;
a gas control subsystem comprising a CO₂ sensor within the enclosure and an electric valve through which the interior of the enclosure is in fluidic communication with a gas supply;
a temperature control subsystem comprising a thermal conditioner in thermal contact with the interior of the enclosure and a thermal sensor within the enclosure; and
a microcontroller communicatively coupled to the CO₂ sensor, the electric valve, the thermal conditioner, and the thermal sensor;
wherein the microcontroller is configured to provide a gas control-feedback loop, driving the electric valve in response to the CO₂ sensor;
wherein the microcontroller is further configured to provide a thermal control-feedback loop, driving the thermal conditioner in response to the thermal sensor;
wherein the saturated salt solution of the humidity control subsystem is chosen and the gas control-feedback loop and thermal control-feedback loop are configured such that a characterization loading condition is established and maintained within the interior of the enclosure while the enclosure is sealed.

9. The controlled characterization environment system of claim 8, wherein the saturated salt solution comprises at least one of ammonium nitrate, ammonium sulphate, lithium chloride, magnesium chloride, magnesium nitrate, natrium chloride, potassium sulphate, potassium nitrate, potassium chloride, potassium acetate, potassium hydroxide, sodium chloride, sodium nitrite, and sodium dichromate.

10. The controlled characterization environment system of claim 8, wherein the gas supply is an air line, with the air line putting the interior of the enclosure in fluidic communication with an ambient atmosphere through the electric valve.

11. The controlled characterization environment system of claim 8, wherein the gas supply is one of an air compressor and a gas cylinder.

12. The controlled characterization environment system of claim 8, wherein the characterization loading condition comprises a CO₂ concentration that is between 400-500 ppm.

13. A controlled characterization environment system, comprising:
an enclosure that is sealable and comprises an interior;
a humidity control subsystem;
a gas control subsystem comprising a CO₂ sensor within the enclosure and an electric valve through which the interior of the enclosure is in fluidic communication with a gas supply; and
a microcontroller communicatively coupled to the CO₂ sensor and the electric valve, with the microcontroller configured to function in a gas control-feedback loop, driving the electric valve in response to the CO₂ sensor;
wherein the humidity control subsystem and the gas control-feedback loop are configured such that a characterization loading condition is established and maintained within the interior of the enclosure while the enclosure is sealed.

14. The controlled characterization environment system of claim 13, wherein the humidity control subsystem comprises a vessel inside the enclosure, with the vessel containing a saturated salt solution.

15. The controlled characterization environment system of claim 14, wherein the saturated salt solution comprises at least one of ammonium nitrate, ammonium sulphate, lithium chloride, magnesium chloride, magnesium nitrate, natrium chloride, potassium sulphate, potassium nitrate, potassium chloride, potassium acetate, potassium hydroxide, sodium chloride, sodium nitrite, and sodium dichromate.

16. The controlled characterization environment system of claim 13, wherein the humidity control subsystem comprises a bubbler.

17. The controlled characterization environment system of claim 13, wherein the gas supply is an air line, with the air line putting the interior of the enclosure in fluidic communication with an ambient atmosphere through the electric valve.

18. The controlled characterization environment system of claim 13, wherein the gas supply is one of an air compressor and a gas cylinder.

19. The controlled characterization environment system of claim 13, further comprising a temperature control subsystem comprising a thermal conditioner and a thermal sensor, wherein the thermal conditioner and thermal sensor are communicatively coupled to the microcontroller, and wherein the microcontroller is configured to provide a thermal control-feedback loop, driving the thermal conditioner in response to the thermal sensor.

20. The controlled characterization environment system of claim 13, wherein the characterization loading condition comprises a CO₂ concentration that is between 400-500 ppm.
